(19) United States
(12) Patent Application Publication
(10) Pub. No.: US 2019/0134177 A1
(43) Pub. Date: May 9, 2019

(54) CONJUGATE FOR VACCINATION AGAINST TYPHOID COMPRISING CHEMICAL CONJUGATE OF VI POLYSACCHARIDE AND FLAGELLIN, A PROCESS FOR PRODUCING THE SAME AND A COMPOSITION COMPRISING THE CONJUGATE

(71) Applicant: National Institute of Immunology, New Delhi (IN)

(72) Inventors: Jairam Meena, New Delhi (IN); Amulya Kumar Panda, New Delhi (IN)

(21) Appl. No.: 16/096,387

(22) PCT Filed: Apr. 21, 2017
(86) PCT No.: PCT/IN2017/050142

§ 371 Date: Oct. 25, 2018

(30) Foreign Application Priority Data
Apr. 25, 2016 (IN) 201611002743

(51) Int. Cl. A61K 39/112; A61K 9/00; A61K 47/54; A61K 47/32; A61K 47/34; A61K 47/69; A61P 31/04

(52) U.S. Cl. CPC A61K 39/0275; A61K 9/0019; A61K 47/542; A61K 2039/55555; A61K 47/34; A61K 47/6905; A61P 31/04; A61K 47/32

(57) ABSTRACT

The present investigation relates to a conjugate comprising flagellin adjuvant covalently linked to Vi polysaccharide derived from S. typhi for vaccination against typhoid. Both flagellin adjuvant and Vi polysaccharide are from S. typhi which leads to the improved immunogenicity. The conjugate of the present invention can be used as single dose administration without the need of multiple immunizations. The present invention also discloses a nanoparticle composition comprising the conjugate of the present invention.

### FIELD OF INVENTION

The present invention pertains generally to the field of vaccine development. In particular, the present invention pertains to a novel typhoid vaccine.

### BACKGROUND

Polysaccharide based vaccine are poorly immunogenic, don't induce isotype switching and never elicit memory antibody response. To improve the immunogenicity of polysaccharide based vaccine, protein molecules are conjugated to it. This chemical conjugation improves the immunogenicity of carbohydrate vaccine and elicits memory antibody titers. Conjugate vaccines are type of subunit vaccines which contains a T cell independent polysaccharide antigen conjugated to a T cell dependent antigen. These vaccines produce high affinity IgG antibodies in infants and adults and elicit long lasting antibody response as compared to vaccines based on only polysaccharides. Many conjugate vaccines are thus developed for bacterial infection like typhoid, pneumoniae and influenza.

In most of the cases of conjugate vaccine, carbohydrate is conjugated to a peptide which provides T cell help. It will be ideal if peptide of the same pathogen which has been considered as a candidate vaccine is conjugated to the polysaccharide. It will not only alter the T cell independency of the carbohydrate but also will provide immunological help by eliciting antibody against the protein molecule. Moreover, if the protein of interest used for conjugation is a ligand for TLR, it further helps in improving the immunogenicity of the conjugate vaccine.

Salmonella typhi (S. typhi) is the most common intracellular pathogen. Among 2,300 closely-related serovars, S. typhi and typhimurium are exclusively pathogenic to humans, causing typhoid or enteric fever and gastroenteritis (Res. Microbial. 2004. 155:568-570, Infect. Immun. 66 (1998) 4579-4587). Though certain antibiotic treatments have been indicated for typhoid, none of the antibiotic treatments are free of side effects and such treatments often ineffective for carriers with gallstones (J. Bacterial. 184 (2002) 1270-1276, Infect. Immun. 70 (2002) 2640-2649) and therefore there is a need for vaccine.

In the last decade, conjugate vaccines have been developed for typhoid disease. For instance, a safe and highly immunogenic conjugate vaccine based on Vi (polysaccharide from Salmonella enterica serovar typhi) and rEPA protein carrier was developed by NICHD/NIH (Lanh et al., N. Eng. J. Med. 2003; Thiem et al., Clin Vac. Immunol. 2011; Szu, Expert Rev. Vaccines 12(11), 1273-1286 (2013)). A number of papers discuss the immunogenicity of Vi, its conjugate vaccines and the Vi chain length considered hitherto optimal (Szu et al., Infection and Immunity, 1989, 3823; Szu et al., Infection and Immunity, 1991, 4555; Szu et al. Infection and Immunity, 1994, 5545; Kossaczka et al., Infection and Immunity, 1999, 5806; Cui et al., Clin. Vaccine Immunol., 17 (2010), 73-79; Micali et al., Vaccine, 29 (2011), 712-720; An et al., Vaccine, 29 (2011), 7618-23; Rondini et al., Clin. Vaccine Immunol., 18 (2011), 460-68; An et al., Vaccine, 30 (2012), 1023-1028).

There are certain typhoid vaccines known in the prior art, For instance, WO 1996/011709 discloses an O-acetylated oligonucleotide or polygalactouronate pectin which is substantially identical to Vi polysachharide subunit structure conjugated to a carrier protein tetanus toxoid wherein the carrier protein being derivatized with cystamine. This particular patent teaches conjugation of an identical polysaccharide.

WO 1998/026799 discloses an isolated lipo-polysachharide from Salmonella Paratyphi A, having removed its Lipid A through detoxification and retaining its O-acetyl content between 70% to 80% and then conjugated to a carrier protein tetanus toxoid through adipic acid dihydrazide (ADH). WO2000/033882 discloses a Vi-polysaccharide of the Salmonella typhi covalently bound to a protein pseudomonas aeruginosa (Vi-rEPA) conjugate through adipic acid dihydrazide. WO2007/039917 discloses an exogenous antigen of Salmonella typhi which is covalently/non-covalently bonded to a Heat Shock Protein. WO2009/150543 describes a conjugated Vi-polysachharide to be used as a vaccine composition against Salmonella typhi causing typhoid fever, wherein the Vi-polysaccharide is covalently conjugated to a protein selected from CRM197 or tetanus toxoid. The method of conjugation as disclosed in WO2009/150543 includes first simultaneously adding carrier protein which is preferably CRM197 or tetanus toxoid to a linker such as adipic acid-dihydrazide (ADH), and a carbodiimide such as I-ethyl-3(3-dimethylaminopropyl) carbodiimide (EDAC), to give a derivatized carrier protein in presence of a 2-(N-morpholino) ethane sulphonic acid (MES buffer). The weight ratio of the carbodiimide EDAC to the carrier protein is between 0.1 to 0.15. It also discloses that higher amounts of carbodiimide/protein ratios can cause aggregate formation. Derivatization of the carrier protein is followed by activation of the Vi-polysachharide (ViPs) as well. The Vi-polysaccharide is also activated with a carbodiimide wherein various ratios of ViPs and carbodiimide (EDAC) are mixed to activate the Vi-polysaccharide. It is mentioned that Vi activation can be performed at room temperature within 2 minutes wherein higher ratios between 1.5:1 to 200:1 can be used. The derivatized carrier protein CRM197 or tetanus toxoid and the activated Vi-polysaccharide of Salmonella typhi is then reacted with each other to get the conjugated ViPs-CRM197 or ViPs-TT conjugate, followed by removal of the excess linker.

More recently, a Salmonella typhi vaccine conjugate based on Vi from purified Citrobacter freundii sensu lato and CRM197 protein carrier has been described by Micali et al. Vaccine 2012 and Rondini et al., J. Infect. Dev Ctries, 2012. When tested in humans, Vi-CRM197 conjugate vaccine provided higher anti-Vi antibody responses compared to unconjugated Vi after a single immunization and at a lower dose (van Damme et al., PlosOne 2011; further results presented at the 8th International Conference on Typhoid Fever and Other Invasive Salmonelloses, Bangladesh, March 2013). However, the anti-Vi response following revaccination was lower than the primary response and anti-Vi persistence was shorter than desired.

Therefore, there is still a need to provide improved conjugate vaccines, which would reduce cost to patient, and the number of injections which is capable of eliciting sufficient immune response, which simplifies or eliminates other associated technical concerns in the field of conjugation chemistry which is less time consuming, cost-effective and safe.

An object of the invention is to develop a novel conjugate for vaccination against typhoid comprising chemical conjugate of Vi polysaccharide and flagellin, a process for producing the same and a composition comprising the conjugate.

### SUMMARY OF THE INVENTION

The present invention provides a conjugate comprising flagellin adjuvant covalently linked to Vi polysaccharide derived from S. typhi for vaccination against typhoid.

The present invention also provides a process of producing the conjugate comprising the steps of:
i. derivatization of r-flagellin;
ii. activation of S. typhi Vi capsular polysaccharide;
iii. conjugation of derivatized r-flagellin with activated Vi capsular polysaccharide resulting in formation of Vi-flagellin conjugate using cross-linking agents, optionally using homobifunctional cross-linking agents; and
iv. optionally, purification of Vi-flagellin conjugate.

The present invention also provides a nanoparticle composition comprising the conjugate of the present invention, use of conjugate for immunization against typhoid.

The present invention also discloses achieving the desired titre and immunization level based on single administration of the conjugate

### BRIEF DESCRIPTION OF DRAWINGS

The following description given by way of example, but not intended to limit the invention solely to specific embodiments described may be understood in conjunction with the accompanying figures in which:

FIG. 1 depicts Vi polysaccharide and r-flagellin conjugate (Vi-F conjugate) cross-linked by adipic acid dihydrazide.

FIG. 2 depicts size exclusion chromatogram of Vi-flagellin conjugate, Vi polysaccharide and r-flagellin separated by Sephacryl S-1000 column recorded at 280 nm.

FIG. 3 depicts primary and memory IgG antibody response in BALB/c mice immunized with Vi polysaccharide antigen, Vi-flagellin conjugate and antigen entrapped nanoformulations: Vi antigen entrapped nanoparticles, Vi-flagellin conjugate entrapped nanoparticles and Vi TT Typhbar® conjugate vaccine.

FIG. 4 depicts opsonization (1 hour) and clearance (24 hours) of Salmonella typhi using THP-1 macrophage cells with sera (4 weeks) of BALB/c mice immunized with Vi polysaccharide antigen, Vi-flagellin conjugate, Vi antigen entrapped nanoparticles, Vi-flagellin conjugate entrapped nanoparticles and Vi TT Typhbar® conjugate vaccine.

### DESCRIPTION OF THE INVENTION

The present invention pertains to a novel conjugate vaccine, which involves conjugation of flagellin with Vi polysaccharide.

In the design of vaccines, one of the major considerations is to overcome the low immunogenicity of the antigen. The common approaches to this issue are the coupling of the antigen to an immunogenic carrier or adjuvant. The critical issue concerning adjuvants is their toxic effects, whether the adjuvant is immunogenically capable of inducing a neutralizing response against itself and whether it will be approved for human use. The present invention is based, in part, on flagellin adjuvants that enhance immune responses directed against Salmonella typhi, in particular, to enhance immune responses to polypeptide antigens (e.g., PspA) and capsular polysaccharide from S. typhi.

The present invention provides a novel conjugate comprising flagellin adjuvant covalently linked to Vi polysaccharide derived from S. typhi for vaccination against typhoid.

Flagellin proteins are known and described, for example, in U.S. Pat. Nos. 6,585,980, 6130,082; 5,888,810; 5,618,533; 4,886,748 and U.S. Patent Publication No. US 2003/0044429 A1; and Donnelly et al., (2002) J. Biol. Chem. 43: 40456. Most gram-negative bacteria express flagella, which are surface structures that provide motility. The flagella are formed from a basal body, a filament, and a hook that connects the two. The filament is formed of a long polymer of a single protein, flagellin, with a small cap protein at the end. Polymerization of flagellin is mediated by conserved regions at the N- and C-termini, whereas the intervening hypervariable region of the flagellin protein is very diverse in sequence and length among species.

The flagellin can be derived from flagellins from any suitable source. A number of flagellin genes have been cloned and sequenced (see, e.g., Kuwajima et al., (1986) J. Bact. 168:1479; Wei et al., (1985) J. Mol. Biol. 186:791-803; and Gill et al., (1983) J. Biol. Chem. 258:7395-7401). Non-limiting sources of flagellins include but are not limited to S. enteritidis, S. typhimurium, S. dublin, H. pylori, V. cholera, S. marcesens, S. flexneri, S. enterica, T. pallidum, L. pneumophila, B. burgdorferei, C. difficile, A. tumefaciens, R. meliloti, B. clarridgeiae, R. lupine, P. mirabilis, B. subtilis, P. aeruginosa, and E.coli. The present invention preferably utilizes the flagellin sourced from S. typhi.

The Vi-antigen is a capsular polysaccharide of Salmonella typhi. Vi polysaccharide of the present invention can be obtained from Salmonella typhi. It is liner homopolymer of (1-4)-alpha-D-GalApNAC variably O-actylated at e.sub,3(fig.I) (1,5). Whitiriside and Baker in J. Immunol. 86:538-542(1961) and I-andy et al. AM. J. Ilyg. 73:55-56(1961) disclose that the)-acetyl groups on Vi-antigen is essential for its antignicity.

The present invention discloses the conjugation comprising a flagellin adjuvant and one more polypeptide antigen from S. typhi. The antigen component of a fusion protein can be chemically conjugated to flagellin components. Chemical conjugation (also referred to herein as “chemical coupling”) can include conjugation by a reactive group, such as a thiol group (e.g., a cysteine residue) or by derivatization of a primary (e.g., a amino-terminal) or secondary (e.g., lysine) group.

Further, in the present invention, the concentration ratio of Vi polysaccharide and r-flagellin can be in the ratio of 1:1to 1:4, preferably 1:2 and the Vi polysaccharide is conjugated with r-flagellin protein which results in conjugation of flagellin to Vi polysaccharide at carboxylic group through cross-linking agent.

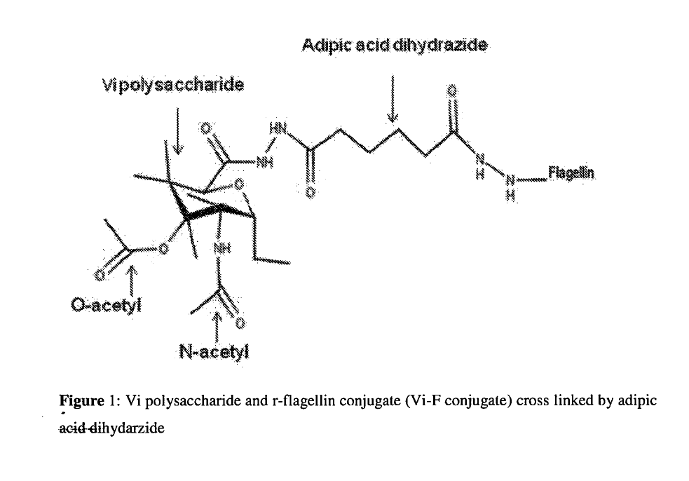

In one embodiment, the Vi polysaccharide is conjugated with r-flagellin protein which results in conjugation of flagellin to Vi polysaccharide at carboxylic group through adipic acid dihydarzide cross-linking agent as shown below at FIG. 1:

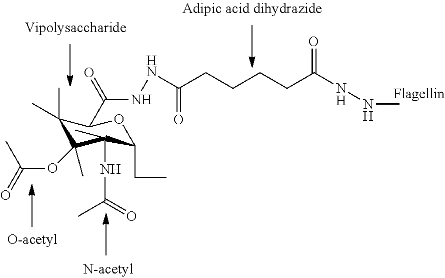

In another embodiment, the present invention provides a process of producing the conjugate comprising the steps of:
i. derivatization of r-flagellin;
ii. activation of S. typhi Vi capsular polysaccharide;
iii. conjugation of derivatized r-flagellin with activated Vi capsular polysaccharide resulting in formation of Vi-flagellin conjugate using cross-linking agents, optionally using homobifunctional cross-linking agents; and
iv. optionally, purification of Vi-flagellin conjugate.

(i) Derivatization of r-flagellin

The derivatization of r-flagellin reaction can be carried out in presence of buffer such as 2-(N-morpholino)ethanesulfonic acid (MES) followed by addition of by addition ofadipic acid dihydrazide (ADH) and subsequently I-ethyl-3-(3-dimethylaminopropyl) carbodiimide (EDAC).

The ratio of adipic acid dihydrazide and r-flagellin can be in the range of 2.5% to 5% w/w ADH/r-flagellin and I-ethyl-3-(3-dimethylaminopropyl) carbodiimide and r-flagellin (EDAC/r-flagellin) ratio can be in the range of 0.15% to 0.3% w/w.

A buffer, such as 2-(N-morpholino) ethanesulfonic acid (MES), can be added to the solution containing the carrier protein prior to the addition of ADH and EDAC. The weight ratio of the carbodiimide to the protein is typically 0.1 to 0.15, as higher carbodiimide/protein ratios can cause aggregate formation. Following the derivatisation of the carrier protein, any excess linker (e.g. ADH) can be removed by, for example, dialysis or tangential flow filtration (TFF).

(ii) Activation of S. typhi Vi Capsular Polysaccharide

Vi isactivated with a carbodiimide and can be subsequently combined with the derivatised earner protein. The activation of S. typhi Vi capsular polysaccharide can be carried out m the presence of 2-(N-morpholino)ethanesulfonic acid (MES) m the presence of I-ethyl-3-(3-dimethylaminopropyl) carbodiimide (EDAC). The ratio of Vi and I-ethyl-3-(3-dimethylaminopropyl) carbodiimide (Vi:EDAC) can be in the range of 1:2 to 1 wt/wt. For Vi activation, various ratios of Vi and carbodiimide can be used. A 1:1 molar ratio (COOH groups of Vi to carbodiimide) can be used, but to reduce the amount of residual unconjugated carbodiimide derivatives (e.g. ureas such as EDU; N-ethyl-N-(3-dimethylaminopropyl)urea, a soluble reaction product of EDAC coupling) higher ratios can be used i.e. with a molar excess of Vi e.g. >1.5:1 and ideally D 3:1, such as 3:1, 4:1, 5:1, 6:1, 7:1, 8:1, 9:1 or higher. Ratios up to 200:1 might be used. These ratios can be considered higher than the ones used in reference 3. Vi activation can be performed at room temperature e.g. in about 2 minutes.

(iii) Conjugation of Derivatized r-Flagellin With Activated Vi Capsular Polysaccharide

The conjugate between the flagellin protein and Vi polysaccharide is obtained by mixing the protein and the polysaccharide together at room temperature and enabling them to react and for the conjugate. Formed Vi polysaccharide protein conjugate can be separated using size exclusion chromatography such as Sephacryl S-1000 column and the conjugate can be eluted much before the unconjugated polysaccharide and unconjugated flagellin can be dried using lyophilized. The conjugated product can be characterized in detail and used for immunization. It is observed that the novel conjugate of the present invention gives higher antibody titer in comparison to Vi polysaccharide alone. The conjugation reaction can be carried out in pH range of 5.4 to 5.8.

The cross-linking agents of the present invention can be selected from the group comprising carbodiimides such as 1-cyclohex yl-3-(2-morpholinyl-(4-ethyl)carbodiimide (CMC), I-ethyl-3-(3-dimethylaminopropyl)carbodiimide (EDC), and I-ethyl-3-(4-azonia-44-dimethylpentyl)carbodiimide. Other suitable cross-linking agents can be selected from the group comprising cyanogen bromide, glutaraldehyde and succinic anhydride. In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative or a homobifunctional photoreactive compound can be used. Also included are heterobifunctional compounds, for example, compounds having an amine-reactive and a sulfbydryl-reactive group, compounds with an amine-reactive and a photoreactive group, and compounds with a carbonyl-reactive and a sulfbydryl-reactive group.

Specific examples of homobifunctional cross-linking agents may include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfbydryl-reactive cross-linkers 1,4-di-[3-(2-pyridyldithio)propionamido]butane, bismaleimidohexane, and bis-N-maleimido-1,8-octane; the bifunctional aryl halides I,5-difluoro-2,4-dinitrobenzene and 4,4-difluoro-3,3-dinitrophenylsulfone; bifunctional photoreactive agents such as bis-[b-(4-azidosalicylamide)ethyl]disulfide; the bifunctional aldehydes formaldehyde, malondialdehyde, succinaldehyde, glutaraldehyde, and adiphaldehyde; a bifunctional epoxied such as 1,4-butaneodiol diglycidyl ether; the bifunctional hydrazides adipic aciddihydrazide, carbohydrazide, and succinic acid dihydrazide; the bifunctional diazoniums o-tolidine, diazotized and bis-diazotized benzidine; the bifunctional alkylhalides N₁N-ethylene-bis(iodoacetamide), N₁N-hexamethylene-bis(iodoacetamide), undecamethylene-bis(iodoacetamide), as well as benzylhalides and halomustards, such as ala-diiodo-p-xylene sulfonic acid and tri(2-chloroethyl)amine, respectively, preferably the reagent is adipic acid dihydrazide (ADH) and EDAC in MES (morpolino ethane sulphonic acid) buffer.

In another embodiment, the present invention provides a composition comprising the novel conjugate of the present invention. The present conjugate can be administered as nanoparticle. The nanoparticles of the present invention can be lipid based nanoparticles, polymer nanoparticles, metallic nanoparticles, surfactant based emulsions. The polymer utilized for preparing the nanoparticles can be selected from the group comprising polylactide, polyglycolide, polylactide-co-glycolide, polycaprolactone, polyhydroxyacid, preferably polylactic acid. The nanoparticle composition of the present invention can be prepared by entrapping the conjugate in a polymer.

In another embodiment, the present invention discloses a process for preparing the nanoparticles composition comprising the steps of:
i. dissolving the conjugate in a solution;
ii. adding an emulsifier such as 1% PVA;
iii. adding an organic phase comprising solvents such as dichloromethane and sonification to form a primary (W/0) emulsion; and
iv. further emulsification of the primary emulsion from step (iii) to obtain a secondary (W/0/W) emulsion.

The method of entrapping conjugate in Polylactic acid nanoparticles of the present invention can be conducted by using any known method in art, preferably by double emulsion solvent evaporation method.

In another embodiment, the present invention discloses use of the conjugate of the present invention for immunization against typhoid and as single dose administration for immunization against typhoid.

Advantage

1. The present invention discloses a novel conjugate vaccine with optimum selection of the carrier protein & immunogen.

2. The present invention discloses novel conjugate of Vi polysaccharide conjugate and flagellin both from S. typhi which leads to the improved immunogenicity.

3. The present invention provides a homologous conjugate.

4. The present invention's novel flagellin-Vi polysaccharideconjugate helps m efficient clearance of intracellular pathogen.

5. The present invention's novel flagellin-Vi polysaccharide conjugate can be used as single dose administration without the need of multiple immunization.

6. The present invention discloses a novel conjugate vaccine which can be administered as nanoparticle.

Selection of carrier protein for conjugation to the capsular polysaccharide is very crucial to elicit desired immune response. Different carrier proteins generate different antibody titers for the same capsular polysaccharide antigen. Use of flagellin as a carrier protein for conjugation to Vi capsular polysaccharide have several advantages. Firstly, flagellin is a TLR 5 agonist thus can strongly activate innate immune cells particularly dendritic cells and macrophages. This will lead to strong T cell activation and better T cell help to Vi polysaccharide (its TLR nature may provide additional adjuvant effect) and might generate memory antibody response. Secondly, use of homologous carrier protein (flagellin from S. typhi) for conjugation to Vi capsular polysaccharide will also generate antibodies against the carrier protein. Therefore, it is expected that the conjugate having dual antigen will elicit anti-Vi and anti-flagellin antibodies, which will eventually work synergistically to offer better protection against S. typhi infection. Further use of PLA nanoparticle based delivery system for Vi flagellin conjugate vaccine improve our understanding towards the use of nanoparticle based adjuvants and delivery system for polysaccharide protein conjugate vaccines.

Without being limited by theory, the Vi polysaccharide antigen mainly elicits IgM antibody response after primary immunization and fails to induce memory antibody response after boosting. Conjugation of Vi polysaccharide antigen with flagellin carrier protein showed antibody class switching and memory antibody response after boosting. Entrapment of Vi-F conjugate inside the PLA nanoparticles shows higher IgG antibody response as compared to Vi polysaccharide, Vi NPs, Vi-F conjugate. As compared Vi-TT Typhbar®, Vi F NPs shows lower IgG antibody titer post boosting. Vi F NPs shows lower anti Vi IgG antibody titer as compared to Vi TT Typhar®, even though the S. typhi binding affinity, opsonization and clearance of Vi F NPs is better than Vi TT Typhbar®. S. typhi binding affinity, opsonization and clearance of Vi NPs and VI-F conjugate are similar. Binding affinity, opsonization and clearance of anti flagellin antibodies are very less and nonsignificant as compared to saline treated mice. Entrapment of Vi-F conjugates inside the PLA nanoparticles results higher antibody titer and class switching. Entrapment of Vi PS or Vi-F conjugate inside the PLA nanoparticle results in inhibition of IgG3 subtype and enhanced the IgG1 and IgG2 subtypes. Enhanced anti Vi IgG response results due to the use of PLA nanoparticles as an adjuvant and antigen carrier along with the use of flagellin as TLR5 agonist. It is also observed that antibody class switching and memory antibody response in case of Vi PLA NP and Vi F NPs, class switching in case of Vi-F NPs is due to the use of flagellin carrier protein which results in processing and presentation of polysaccharide epitop to T cells along with the protein component which provides T cells help to B cells and results in antibody class switching and memory antibody response and the entrapment of polysaccharide antigen in PLA nanoparticles results in class switching and memory antibody response, in case of Vi PLA nanoparticle, class switching and memory antibody response is due to multiple display of antigenic epitop on PLA nanoparticle surface and there extensive cross-linking with B cell receptor along with the enhance cytokine secretion.

The present invention will now be described in more detail with reference to the examples and accompanying drawings, in which preferred embodiments of the invention are shown. This invention may, however, be embodied in different forms and should not be construed as limited to the embodiments set forth herein.

### EXAMPLES

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. Adipic acid dihydrazide (ADH), I-Ethyl-3-(-3-dimethylaminopropyl) carbodiimide hydrochloride (EDAC), 2-(N-morpholino) ethanesulfonic acid (MES), Sodium chloride (NaCl), Sodium hydroxide (NaOH), Sodium phosphate monobasic monohydrate (NaH₂PO₄—H₂O), concentrated hydrochloric acid (cone. HCl), Polylactic acid (PLA), Polyvinyl alcohol (PVA), Dichloromethane (DCM), Acetonitrile (ACN), Tween 20, Sulphuric acid (H₂SO₄).

### Example 1

### Preparation of Conjugate Vaccine of the Present Invention

### Example 1.1

### Purification of r-Flagellin

E. coli BL21 cells are transfected with Pet3d vector encoding Salmonella typhi. Transfected cells are cultured overnight in modified LB (Luria-Bertani) broth containing 5 g/1 glucose (37° C., 200 rpm), supplemented with 100 μg/mL ampicillin. The culture is grown overnight and diluted with fresh modified LB broth containing ampicillin (100 μg/ml) and further allowed to grow until OD₆₀₀ reached to 0.5. The culture is then induced with 1 mM isopropyl-D-1-thiogalactopyranoside (IPTG) and again allowed to grow for 3 hr at 37° C. at 200 rpm. The cell pellet obtained from 2 L culture is processed for protein purification by NI NTA column and analyzed for r-flagellin expression by SDS page. Recombinant flagellin is purified using Ni NTA column. Most of the column bound flagellin eluted at 50 and 100 mM imidazole gradients. 100 mM imidazole eluate contains the pure single band of flagellin at 52 kDa. The amount of r-flagellin is quantified by micro BCA in dialyzed and lyophilized fraction of 100 mM imidazole gradient. The amount of r-flagellin in dialyzed and lyophilized elute is found 350 μg/mg. The eluted flagellin (100 mM) after dialysis and lyophilization is tested for its stability.

### Example 1.2

### Derivatization of r-Flagellin

For the derivatization of r-flagellin, 10 mg of lyophilized r-flagellin is dissolved in 1 ml 80 mM 2-(N-morpholino) ethanesulfonic acid buffer (MES buffer, pH 5.6) followed by addition of adipic acid dihydrazide (ADH/r-flagellin, 2.5% w/w), subsequently I-ethyl-3-(3-dimethylaminopropyl) carbodiimide (EDAC/r-flagellin, 0.15% w/w) is added until solution is clear. Final volume of derivatization reaction is 1 ml, and the final concentration of r-flagellin, ADH and EDAC was 10 mg/ml, 25 mg/mL (200 mM) and 1.5 mg/ml (21 mM), respectively. The reaction is allowed to proceed at room temperature for 90 min at pH 5.6 (pH maintained by adding 1 M HCl). At the end of 90 min, reaction is stopped by bringing pH around 7.4 by addition of 1 M NaOH. Unbound ADH and residual EDAC are removed by dialysis against 400 volume of 80 mM MES buffer using a 20 kDa dialyzing membrane.

### Example 1.3

### Activation of S. typhi Vi Capsular Polysaccharide

Salmonella typhi Vi capsular polysaccharide is activated for conjugation with derivatized r-flagellin. Briefly, 5 mg of Vi capsular polysaccharide is dissolved in 1 ml of 80 mM MES buffer (pH 5.6) subsequently 10 mg of EDAC is added into 1 ml of it (Vi:EDAC, 1:2 wt/wt) and-allowed to react for 10 min at room temperature with continuous stirring.

### Example 1.4

### Conjugation of Derivatized r-Flagellin With Activated Vi Capsular Polysaccharide

Conjugation of Vi polysaccharide to activated r-flagellin is carried using process described in FIG. 1. Conjugation is carried out at different protein to polysaccharide ratios. Activated r-flagellin is added in different r-flagellin/Vi polysaccharide ratios (wt/wt) as following 2:1, 1:1, 1:2, 1:3, 1:4 to get variable amounts of protein in different reaction lots of Vi capsular polysaccharide. In all the reaction lots, concentration of activated Vi polysaccharide is kept constant (5 mg) and amount of activated r-flagellin is varied. The total reaction volume is kept constant, 2 ml (2.5 mg/ml Vi) in all reactions.

Final concentration of Vi polysaccharide to r-flagellin (Vi PS/r-flagellin, wt/wt) 4.0, 2.0, 1.0, 0.5, 0.25 were used for conjugate formation. Conjugates with higher r-flagellin concentration were also attempted, however due to high concentrations of EDAC and ADH in MES buffer during the derivatization of r-flagellin resulted in aggregation hence Vi polysaccharide to r-flagellin ratios are used for Vi flagellin glycoconjugate formation as mentioned in table 1. The activated Vi polysaccharide was conjugated with derivatized r-flagellin protein which results in conjugation of flagellin to Vi polysaccharide at carboxylic group through NHS linker (FIG. 1).

Conjugation of activated Vi polysaccharide with earner proteins being flagellin is optimized at different protein to polysaccharide ratios as mentioned in table I below.

TABLE I
Conjugation conditions used for generation
of different Vi-flagellin conjugate lots

| | | | | Reaction | | |
|---|---|---|---|---|---|---|
| | | Vi/F | Vi/F | time | For Vi | Vi/F |
| | Vi | ratio | ratio in | (min) for | activa- | conju- |
| Conju- | cone. | (w/w) in | conjugate | flagellin | tion | gation |
| gates | (mg/ml) | reaction | (w/w) | activation | (min) | (min) |
| Lot1 | 2.5 | 4:1 | 14.76 | 90 | 5 | 240 |
| Lot2 | 2.5 | 2:1 | 8.75 | 90 | 5 | 240 |
| Lot3 | 2.5 | 1:1 | 1.56 | 90 | 5 | 240 |
| Lot4 | 2.5 | 1:2 | 0.76 | 90 | 5 | 240 |
| Lot5 | 2.5 | 1:4 | 0.62 | 90 | 5 | 240 |
| Lot6 | 2.5 | 1:6 | — | 90 | 5 | 240 |
| Lot7 | 2.5 | 1:10 | — | 90 | 5 | 240 |
| Lot7 | 2.5 | 1:10 | — | 90 | 5 | 240 |

From the table 1 above, it can be noted that. Vi polysaccharide to carrier protein ratio higher than 1:2 shows extensive cross-linking of protein and Vi polysaccharide and thus resulting in viscous and turbid preparation while Vi polysaccharide to r-flagellin protein ratio at 1:6 results in highly viscous solid gel formation. Therefore only 1:1 and 1:2 conjugation ratios are selected (wt/wt, 5 mg Vi polysaccharide with 5 and 10 mg r-flagellin) for further investigations mentioned as Lot 3 and Lot 4 in table 1). Polysaccharide to protein ratios below 1:1 (wt/wt) shows very less protein conjugation to Vi polysaccharide antigen. Hence, on the basis of preliminary immunization studies, Vi polysaccharide to r-flagellin 1:2 ratio is selected for further immunization and nanoparticle formation.

### Example 1.5

### Purification of Vi-Flagellin Conjugate by Size Exclusion Chromatography (SEC)

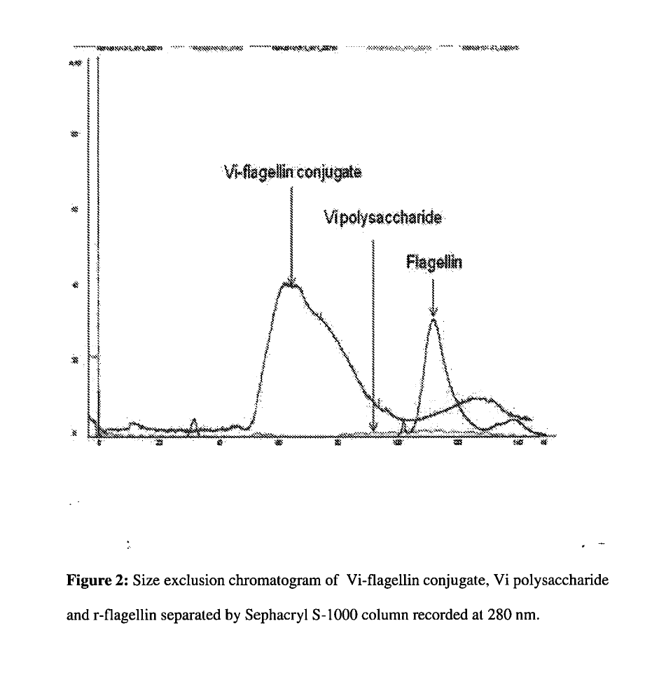

The optimal conjugate lots are dialyzed against 1000 volumes of potassium phosphate buffer (10 mM, containing 100 mM NaCl, pH 7.4) using 300 kDa dialysis membranes to remove free (unconjugated) EDAC, ADH and r-flagellin into it. Resultant dilate is subjected to size exclusion chromatography to separate conjugated Vi capsular polysaccharide from unconjugated polysaccharide and protein (AKTA Basic equipped with S-1000 column, UV visible spectrophotometer and frac 900 auto fraction collector). For separation, conjugates are resolved using Sephacryl 5-1000 column (16/90) connected with UV visible spectrophotometer and 5 ml injection loop. All the conjugates are centrifuged at 5000 rpm for 2 min and the resultant supernatant (2 mL) is loaded into the column pre-equilibrated with 10 mm potassium phosphate buffer and 100 mM NaCl, pH 7.4, and resolution is done at 0.3 ml/min flow rate. Fractions of eluted conjugates are collected and analyzed for protein to polysaccharide ratios. Eluted conjugates are analyzed at 280 and 215 nm wavelengths for the determination of protein and peptide content in the eluted Vi polysaccharide protein glycoconjugate. Vi polysaccharide antigen, r-flagellin protein and different Vi flagellin conjugates are separated using size exclusion chromatography (AKTA basic protein purifier equipped with UV visible spectrophotometer detector and frac 900 auto fraction collector). Sephacryl S-1000 column is used for the separation of Vi flagellin conjugate from unconjugated polysaccharide and r-flagellin protein. Vi polysaccharide antigen is a high molecular weight (2000 kDa) polymer made of N-acetyl galactosamineuronic acid monomer unit. Vi polysaccharide eluted around at 65-95 ml. As compared to Vi polysaccharide, Vi flagellin conjugate, unconjugated protein are eluted around at 45-65 ml and 105-110 ml, respectively (FIG. 2). Elution chromatogram at 280 nm shows that Vi-F conjugate eluted much earlier as compared to Vi polysaccharide. Elution of Vi flagellin conjugate much earlier and high absorbance of elute at 280 nm and 215 nm shows that Vi polysaccharide is conjugated with r-flagellin carrier protein. Elution profile of Vi polysaccharide shows very less absorbance or even a baseline elution from 65-95 mL, which shows that Vi polysaccharide is devoid of high amount of protein or peptide. Further elution of Vi polysaccharide (65-95 mL) after the elution of Vi flagellin conjugate (45-65 mL) shows that Vi flagellin conjugate is of higher molecular weight as compared the Vi polysaccharide alone. Elution chromatogram of r-flagellin protein shows a sharp high intensity peak between 105-110 mL. Elution of flagellin much later as compared to Vi polysaccharide and Vi-F conjugate shows that flagellin has lower molecular weight as compared to the Vi polysaccharide and Vi flagellin conjugate. Size exclusion chromatogram of Vi flagellin conjugate shows that Sephacryl S-1000 column successfully separated the Vi flagellin conjugate from unconjugated Vi polysaccharide and flagellin protein. 5 ml fractions of Vi flagellin conjugate is collected to minimize the presence of unconjugated Vi polysaccharide in the Vi flagellin conjugate. Fractions with maximum Vi flagellin conjugate are pooled and dialyzed against 1000 volume of PBS (50 mM, pH 7.4) and lyophilized and characterized for protein to polysaccharide ratio and stability and stored at 4° C. for further use.

### Example 1.6

### Estimation of r-Flagellin in Vi-Flagellin Conjugate

Different Vi flagellin conjugates are characterized for flagellin concentration in different lots using micro BCA method. Briefly, 1 mg of Vi flagellin conjugate from each Vi flagellin lot is dissolved in 1 ml of 1% SDS in Milli Q water. Bovine serum albumin is used as standard protein in 5-100 μg/ml concentration range. Amount of flagellin present in different Vi flagellin lots are quantified by absorbance at 562 nm wavelength and calculated using standard graph generated for the BSA at the same wavelength. Absorbance of blank is subtracted from samples and standard BSA. Protein concentration in different Vi flagellin conjugates is determined.

### Example 1.7

### Estimation of Vi Polysaccharide Concentration in Vi-Flagellin Conjugate

Concentration of Vi polysaccharide antigen conjugated with per mg of flagellin protein or amount of polysaccharide antigen conjugated with carrier protein is determined using ELISA. Briefly, 100 μl of anti Vi monoclonal antibodies (MAb) are coated in high binding RIA NUNC 96 well ELISA plate. For coating anti Vi sera is diluted (1:5 ratio) in 100 mM bicarbonate coating buffer (pH 9.6) and incubated for 10 hr at 4° C. After MAb coating and incubation, plate is washed extensively with washing buffer three times and blocked with 1% skim milk powder in washing buffer and incubated for 2 hr at 37° C. After 2 hr of incubation, plate is further washed with washing buffer and incubated with known concentration of Vi polysaccharide antigen (200-5 pg/ml, Vi TT Typhbar® or Vi Typhim) in duplicates in bicarbonate coating buffer for 4 hr at 37° C. For estimation of Vi polysaccharide antigen in Vi flagellin conjugate, Vi flagellin conjugate was (1 mg/ml solution is serially diluted in the same plate) also incubated in the same plate in duplicates. Again plate is washed extensively three times with washing buffer, and incubated with HRP conjugated secondary anti mouse Ig antibodies (1:5000 in bicarbonate buffer) for 1 hr at 37° C. Plate is washed extensively three times with washing buffer and incubated with 100 μl o-phenylenediamine dihydrochloride (OPD) substrate for 10 min at room temperature. Reaction is stopped using 2N H₂SO₄ and color intensity is measured at 563 nm. Absorbance of blank is subtracted and concentration of Vi antigen in Vi flagellin conjugate is calculated using 4 parametric logistic log equation using graph pad prism 6.

### Example 1.8

### Estimation of r-Flagellin and Vi Polysaccharide in Vi-F Conjugate

Carrier protein to polysaccharide ratio in glycoconjugate vaccines play important role in its immunogenicity. Protein to polysaccharide ratio in different Vi flagellin glycoconjugate lots were analyzed. As the ratio of flagellin increased for conjugation in reaction mixture, amount of flagellin conjugated with Vi polysaccharide increase. Extent of activation of flagellin with ADH (adipic acid dihydrazide) in presence of EDAC also governs the amount of flagellin linked to the Vi polysaccharide antigen. From 1:1 ratio (r-flagellin:ADH, wt/wt) to 1:3 ratio, flagellin activation increased and hence conjugation of flagellin to the Vi polysaccharide increased. Further increment in ADH ratio and thereby flagellin activation leads into flagellin aggregation. Hence in all of the conjugation lots, which were used for immunization, r-flagellin activation was carried out at 1:2.5 ratios (r-flagellin: ADH, wt/wt). The amount of r-flagellin in a conjugate, prepared by the ratio of 4:1 of Vi polysaccharide: r flagellin, wt/wt) was 16.50 percent, 1 mg of Vi polysaccharide conjugated with 0.33 mg of r-flagellin protein. The amount of r-flagellin conjugated with Vi polysaccharide at higher protein ratio (Vi polysaccharide-flagellin, 1:4, wt/wt) was much higher as compared to the lower protein ratio and it was 1.62 mg protein conjugated with 1 mg Vi polysaccharide. As the amount of conjugated protein increases in the Vi flagellin conjugate, viscosity of the conjugate increases. At higher protein concentration (Vi polysaccharide: r-flagellin ratio, greater than 1:4), higher cross-linking of the protein and polysaccharide results into glycoconjugate which is highly viscous and unable to load into the Sephacryl S-1000 column for chromatographic separation.

### Example 1.9

### O-Acetyl Content Estimation in Vi Polysaccharide and Vi-Flagellin Conjugate

Vi polysaccharide 1 s a homo-polymer made-up of repeating units of N-acetyl galactosamineuronic acid which contains O-acetyl at C₃ position while N-acetyl at C₂ position. O-acetyl group at C₃ position in Vi polysaccharide antigen is of prime importance as it governs the immunogenicity and antibody formation. Chemical conjugation of Vi polysaccharide antigen with r-flagellin carrier protein and/or hydrolysis of Vi polysaccharide antigen can change the immunogenicity of antigen. Even different culture conditions of S. typhi culture for Vi production may resulting into a lot to lot variation in O-acetyl content thus immunogenicity of Vi polysaccharide antigen. As O-acetyl content is critical factor for the immunogenicity hence O-acetyl content of Vi polysaccharide antigen and Vi flagellin conjugate were estimated using hestrin method and compared with the Vi polysaccharide antigen for any change in O-acetyl content [10].

### Example 2

### Illustration of the Composition of the Present

### Example 2.1

Recombinant flagellin, Vi capsular polysaccharide and Vi-F conjugate entrapped PLA NPs were prepared by double emulsion solvent evaporation method with slight modification (15). Briefly, antigens were dissolved in 400 μl of aqueous phase (Wl) containing 0.5% (w/v) PVA (30-70 kDa) as emulsifier. This Wl phase was added in 4 ml of DCM organic phase (OP) containing 50 mg/ml PLA (0.4 dl/gm) polymer with sonication to form primary emulsion (Wl/O). Primary emulsion was added to external aqueous phase (W2) containing 1% PVA (30-70 kDa) as surfactants with sonication to form water in oil in water (Wl/O/W2) secondary emulsion. Primary emulsion was sonicated with drop by drop addition for 2 minute at 30% watt and 20 duty cycles using Bandelin sonicator (Bandelin, UK) equipped with S-2000 controller and MS-72 probe. Secondary emulsion was formed with drop by drop addition of primary emulsion into W2 phase by sonication at 30% watt and 20 duty cycles for 3 min using the same sonicator and probe. Plain nanoparticle was prepared without addition of antigen in Wl phase. Further, florescent NPs for microscopic studies were prepared by addition of 50 μl of coumarin 6 dye in OP. The NPs formed were kept on magnetic stirring (100 rpm) for 7 to 8 hr at room temperature for evaporation of dichloromethane. After evaporation of OP, NPs were pellet down at 17000 rpm for 15 min (Sorvall RC6+) at 4° C. and washed thrice with Milli Q water to remove the residual PVA. The NPs were then re-suspended in 5 ml Milli Q water and frozen at −80° C., lyophilized and were stored at 4° C. for further use.

### Example 2.2

### Characterization of Antigen Entrapped PLA Nanoparticles

Vi polysaccharide entrapped nanoparticle (Vi PS NP), r-flagellin entrapped nanoparticles (F NPs), Vi polysaccharide flagellin conjugate entrapped nanoparticles (Vi F NP) and plain nanoparticles (D NPs) were characterized for particle size, zeta potential and polydispersity index using Malvern zetasizer. The surface morphology of different antigen entrapped PLA nanoparticles were analyzed by transmission electron microscopy (TEM). For characterization using zetasizer, NPs were re-suspended in Milli Q water (1 mg/ml) and suitably diluted to avoid multi scattering phenomena. Readings were recorded in triplicates for three different nanoformulations (each antigen entrapped PLA nanoparticles) and results were expressed as mean±SEM.

Vi polysaccharide, r-flagellin and Vi-F conjugate entrapped PLA NPs were formulated by double emulsion solvent evaporation method. For nanoparticle formation, internal aqueous phase (IAP) to OP ratios was optimized. In all formulations, PLA concentration was kept constant at 50 mg/ml (4 ml DCM) and different internal aqueous phase ratios were optimized to reduce the nanoparticle size and to improve the antigen load. Internal aqueous phase to organic phase ratios, 1:40, 1:20 and 1:10 (IAP:OP) (100:4000, 200:4000 and 400:4000 μl) were used. The NPs (Vi NPs, Vi-F NPs and F NPs) were characterized for their size, zeta potential and polydispersity index by zeta sizer. Different nanoformulation at IAP:OP ratio 1:40 showed the smallest nanoparticles size, Vi F NPs showed 284.2 nm, whereas Vi NPs showed 290.8 nm size. Zeta potential at the same IAP:OP ratios (1:40) was −14.5 and −14.9 mV respectively for Vi F NPs and Vi NPs. Nanoparticle size at 1:10 ratio was highest. The minimum nanoparticle size was 284.2 nm for Vi-F NPs at 1:40 ratio. Zeta potential of D NPs was −8.92 mV. In case of Vi NPs and Vi-F NPs zeta potential were slightly negative as compared to the D NPs. In case of Vi NPs zeta potential was −14.9 mV. As compared to D NPs, negative deviation of 6.0 mV in Vi NPs zeta potential was due to the presence of antigen at the surface of NPs. Vi polysaccharide contains COOH functional group and it is negatively charge. In case of Vi NPs presence of negatively charge Vi polysaccharide antigen at the nanoparticle surface imparted slightly negative zeta potential as compared to the D NPs. In accordance to the Vi NPs, Vi-F NPs showed similar negative deviation in zeta potential. Zeta potential in case of Vi NPs and Vi-F NPs were similar as both these nanoparticle entrapped the Vi PS antigen. Change in the phase volume ratio did not change the zeta potential of the NPs. The D NPs with different IAP ratios showed the similar zeta potential. Same with the Vi NPs and Vi-F NPs at different IAP ratios which showed similar zeta potential for 100, 200 and 400 μl IAP volumes. These results showed that phase volume ratio changed the nanoparticle size but does not have any effect on zeta potential of NPs. All these nanoparticles, D NPs, Vi NPs and Vi-F NPs showed narrow particle size distribution. Polydispersity index ranges from 0.112 to 0.147 for Vi-F NPs and D NPs. Narrow size distribution of D NPs and different antigen nanoformulation showed that the NPs formed were homogeneous in size. Similar to the Vi NPs and Vi-F NPs, particle size, zeta potential and polydispersity of F NPs (r-flagellin entrapped nanoparticles) were determined. F NPs with similar size, zeta potential and polydispersity were formed.

### Example 3

### Efficacy of the Novel Conjugate of the Present Invention

### Example 3.1

### Animal Immunization

All the animal immunization experiments were performed with approval of Institute Animal Ethical Committee (IAEC) at the end of the Applicant. Animals were maintained according to the guidelines established by the Institute Animal Ethical Committee and experiments were performed according to the guidelines of the ethical committee. 4-6 week old inbreed female BALB/c mice were divided into groups with six mice in each group and kept in animal house of National Institute of Immunology for acclimatization. Mice were immunized with Vi polysaccharide at a dose 5 μg/mouse or different Vi formulations entrapped in PLA nanoparticles weighed equivalent to 5 μg antigen by intramuscular route (IM). 6.2 μg/mouse flagellin antigen (flagellin conjugated to 5 μg Vi polysaccharide antigen) was immunized by intramuscular route. Animals were boosted after 3 months with 115^{th} of primary immunization dose with same route.

### Example 3.2

### Opsonization and Clearance of S. typhi by THP-1 Macrophage Cells

For S. typhi opsonization and clearance, sera of mice immunized with different antigens such as Vi polysaccharide, Vi NPs, Vi-F conjugate, Vi-F NPs, flagellin, flagellin nanoparticles (F NPs) and Vi TT Typhbar® were diluted at 1:100 in PBS. PBS diluted sera were compliment inactivated by heating at 60° C. for 30 min on water bath. THP-1 cells were obtained from the American Type Culture Collection and were differentiated with phorbol 12-myristate 13-acetate as described. After differentiation, cells were seeded in 24-well plates at a density of 5×10⁵ cells per well and incubated overnight in complete RPMI medium without antibiotics. For S. typhi attachment and opsonization, bacteria were added to cells for 1 hour at 4° C. at a multiplicity of infection (MOI) of 10 bacteria per THP-1 macrophage cells and incubated for 1 h at 4° C. Cells were washed three times with 0.5 ml PBS, and cells were lysed with lysis buffer. Number of bacteria opsonized by THP-1 cells was determined by spreading serial 10-fold dilutions on Luria Bertani (LB) agar plates. To determine bacterial uptake, differentiated THP-1 cells were infected with the S. typhi for 1 hat 37° C. at an MOI of 10 bacteria per THP-1 cell. Cells were then washed three times with 0.5 ml PBS. RPMI medium (0.5 ml) containing 0.1 mg/ml gentamicin was then added to the cells for 90 min. THP-1 cells were then washed 3 times with 0.5 ml PBS and lysed in 0.5 ml of sterile water. For bacterial clearance, THP-1 cells were incubated with 10 MOI of S. typhi for 24 hr. After 24 hr THP-1 cells were then washed three times with 0.5 ml PBS. RPMI medium (0.5 ml) containing 0.1mg/ml gentamicin (Gibco) was then added to the cells for 90 min. THP-1 cells were then washed 3 times with 0.5 ml PBS and lysed in 0.5 ml of sterile water. The recovery of bacteria from macrophages was quantified by spreading serial 10-fold dilutions on Luria-Bertani (LB) agar plates containing the appropriate antibiotics. For opsonization and clearance, the difference in the number of bacterial colonies formed after 1 hour of incubation and after 24 hr of incubation was measured. Single cell bacterial colonies were counted after the colony formation and results were expressed with number of colonies mean±SEM for three mice sera in each group and results were compared using one way ANOVA.

### Example 3.4

### Antigen Load and Entrapment Efficiency

Antigen load and entrapment efficiency of different nanoformulations were determined by ELISA. The Vi polysaccharide is a high molecular weight capsular polysaccharide and thus difficult to encapsulate inside the polymer nanoparticles. In case of Vi NPs the antigen load was 1.33 μg/mg and entrapment efficiency was 33.0 percent. For Vi-F NPs the load of polysaccharide antigen was 1.0 μg/mg and entrapment efficiency was 50%. The load of r-flagellin inside the Vi-F NPs was 1.60 μg/mg and entrapment efficiency was 50%. The amount of protein present per mg of Vi-F nanoparticle was 1.6 μg whereas amount of Vi PS presents in per mg of Vi-F NPs was 1.0 μg. Results for antigen load and entrapment efficiency are in close agreement with the amount of Vi PS and r-flagellin present in Vi-F glycoconjugate as determined with ELISA and micro BCA method which showed that 1.0 μg of Vi polysaccharide was conjugated with 1.65 μg of Vi polysaccharide antigen. Likewise the antigen load and entrapment efficiency for F NPs was determined using micro BCA method. The F NPs showed 1.48 μg/mg antigen load and 38% entrapment efficiency. Low antigen load in different antigen entrapped NPs was due to higher molecular weight of Vi PS antigen (−2000 kDa) and r-flagellin protein (52 kDa). As compared to drugs which are low molecular weight compounds, biological macromolecules are difficult to entrap inside the nanoparticles and hence Vi NPs, Vi-F NPs and F NPs showed lower antigen load and entrapment efficiency.

### Example 3.5

### IgG Specific Anti-Vi Antibody Response in Mice

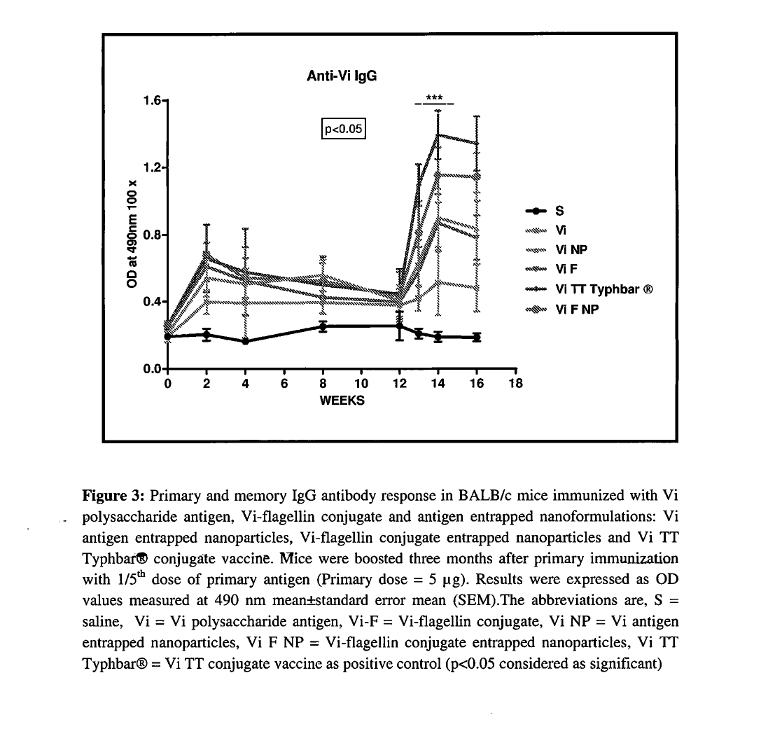

Humoral immune responses against Vi polysaccharide, Vi-F conjugate, Vi TT Typhbar®, Flagellin and polymeric nanoformulation of Vi polysaccharide (Vi NPs), Vi-F conjugate (Vi-F NPs) and flagellin were analyzed in sera of BALB/c mice. Antibody titer was measured using ELISA. Anti-Vi IgG response in all immunized mice was compared and is presented in FIG. 3. Results showed that as compared to saline treated mice, Vi polysaccharide, Vi-F conjugate, Vi NPs, Vi-F NPs and Vi-TT Typhbar® showed significantly higher antibody titers. The anti-Vi antibody responses (anti Vi IgG) from the immunized animals was in the order Vi TT Typhbar®>Vi F NPs>Vi NPs Vi F>Vi polysaccharide. The anti-Vi IgG immune responses in case of Vi NPs and Vi-F conjugate was comparable and was lower than Vi-TT Typhbar® and Vi-F NPs. IgG antibody Immune responses elicited by Vi-TT Typhbar® and Vi-F conjugate entrapped nanoparticles (Vi-F NPs) were also similar. Vi polysaccharide showed highest anti-Vi IgM antibody titers. Conjugation of Vi polysaccharide with carrier protein or entrapment of Vi polysaccharide inside PLA nanoparticles decreased the anti-Vi IgM antibody titers and increased the IgG antibody titer. Higher IgG response in case of Vi-TT Typhbar®, Vi-F conjugate, Vi NPs, Vi-F NPs and lower IgM responses showed that, conjugation of Vi polysaccharide with carrier proteins or entrapment of Vi polysaccharide or Vi-F conjugate inside PLA nanoparticles results in class switching of IgM to IgG. Vi polysaccharide is a T-cell independent antigen and thus fails to class switch from IgM to IgG. Class switching from IgM to IgG in case of glycoconjugates or antigen entrapped nanoformulation indicated that conjugation of Vi polysaccharide with flagellin or entrapment of Vi polysaccharide or Vi-F conjugate inside the polymer nanoparticles provide T cell help to B cells, resulting in IgM to IgG class switching. IgM to IgG class switching was less prominent with Vi NPs and Vi-F conjugate immunized mice when compared with Vi TT Typhbar®. Class switching in case of Vi F NPs was similar to the Vi-TT Typhbar® two weeks post immunization. These results confirm that both conjugation of Vi polysaccharide to carrier protein and its entrapment in PLA nanoparticles leads to class switching of IgM to IgG. This was associated with lower IgM and higher IgG type response. This is important as IgG provides long lasting protection.

### Example 3.6

### Opsonization and Clearance

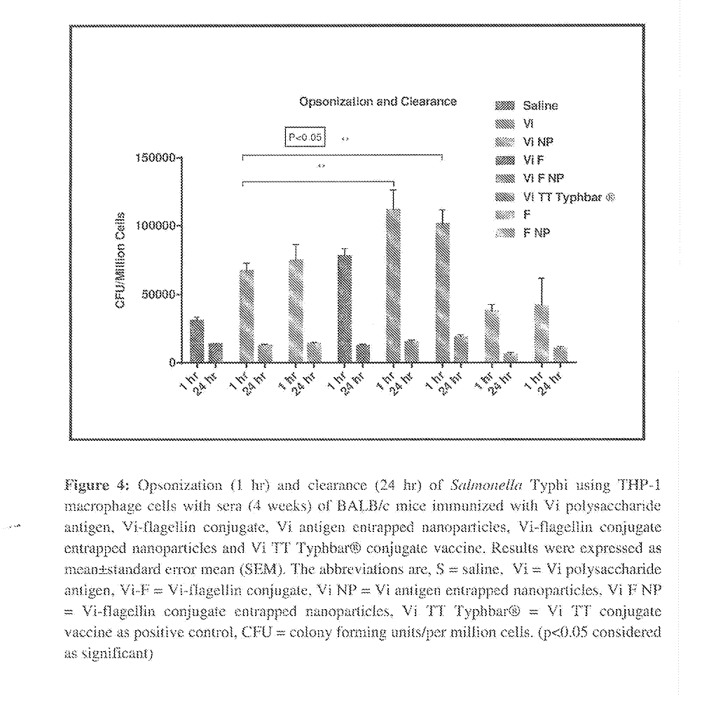

Bacterial opsonization and clearance were analyzed in THP-1 human macrophage cells using sera of mice immunized with different formulations (FIG. 4). Results indicated that as compared to saline treated mice, Vi-F NPs immunized mice showed significantly (P<0.05) higher bacterial opsonization post one hour of infection as well as significantly (P<0.05) higher bacterial clearance post 24 hours of infection (ten folds higher opsonization and clearance). Sera of Vi TT Typhbar® immunized mice also showed significantly higher opsonization of S. typhi one hr post infection as well as post 24 hr of infection (Eight folds higher opsonization and clearance). Mice sera immunized with Vi polysaccharide, Vi NPs and Vi-F conjugate showed similar opsonization post one hr of infection as well as post 24 hr of infection. Opsonization and clearance with mice sera immunized with Vi polysaccharide, Vi-F conjugate and Vi NPs were significantly (P<0.05) higher as compared to saline treated mice (six folds higher). Sera of BALB/c mice immunized with soluble r-flagellin and flagellin entrapped nanoparticles (F NPs) showed slightly higher opsonization one hr post infection as well as clearance post 24 hr of infection (two fold higher opsonization and clearance) (FIG. 4). As compared to Vi TT Typhbar®, mice immunized with Vi-F NPs showed higher opsonization post one hr of infection as well as higher bacterial clearance 24 hr post infection (1.16 fold higher opsonization and clearance) (P<0.05). Total anti-Vi IgG titer in mice immunized with Vi TT Typhbar® and Vi-F NPs showed that as compared to Vi-F NPs, Vi TT Typhbar® showed higher antibody response. Despite the higher anti-Vi IgG response in Vi TT Typhbar® Vi-F NPs showed higher bacterial binding, opsonization and clearance of S. typhi. This higher opsonization and clearance in case of Vi-F NPs as compared to Vi TT Typhbar® showed that entrapment of Vi-flagellin conjugate in nanoparticles further improved the immunogenicity of both Vi polysaccharide and flagellin carrier protein. The anti-Vi antibodies and anti-flagellin antibodies work cooperatively and thus resulted in higher opsonization and clearance as compared to Vi TT Typhbar®. In case of heterologous conjugate (Vi TT Typhbar® in which carrier protein is of different pathogen) only anti-Vi IgG antibodies was binding and resulting in opsonization and clearance whereas in case of homologous conjugate entrapped nanoparticles (Vi-flagellin conjugate entrapped nanoparticle in which both Vi polysaccharide and flagellin carrier protein are from S. typhi) both anti-Vi and anti-flagellin IgG antibodies contributed towards bacterial binding, opsonization and clearance. Vi-F conjugate entrapped nanoparticles (Vi-F NPs) showed significantly higher anti-Vi antibody titer. This is due to the entrapment of Vi-F conjugate inside the PLA nanoparticles which provided the additional adjuvant activity and therefore Vi-F NPs showed significantly higher bacterial binding, opsonization and clearance as compared to Vi-F conjugate alone. These results showed that homologous conjugate (Vi-F conjugate) entrapped nanoparticles showed highest bacterial binding as well as bacterial clearance and can be a better vaccine formulation as compared to Vi TT Typhbar®.

We claim:

1. A novel conjugate comprising flagellin adjuvant covalently linked to Vi polysaccharide derived from S. typhi for vaccination against typhoid.

2. The conjugate as claimed in claim 1, wherein the concentration ratio of Vi polysaccharide and r-flagellin is in the ratio of 1:1 to 1:4, preferably 1:2.

3. A process of producing the conjugate as claimed in claim 1 comprising the steps of:
(i) derivating r-flagellin;
(ii) activating S. typhi Vi capsular polysaccharide;
(iii) conjugating derivatized r-flagellin with activated Vi capsular polysaccharide resulting in formation of Vi-flagellin conjugate using cross-linking agents, optionally using homobifunctional cross-linking agents; and
(iv) optionally, purifying Vi-flagellin conjugate.

4. The process as claimed in claim 3, wherein the derivatization of r-flagellin reaction is carried out in the presence of buffer such as 2-(N-morpholino)ethanesulfonic acid (MES) followed by addition of adipic acid dihydrazide (ADH) and subsequently I-ethyl-3-(3-dimethylaminopropyl) carbodiimide (EDAC).

5. The process as claimed in claim 3, wherein adipic acid dihydrazide is in the range of 2.5-5% w/w in flagellin solution and I-ethyl-3-(3-dimethylaminopropyl) carbodiimide is in the range of 0.15 to 0.3% w/w in flagellin solution.

6. The process as claimed in claim 3, wherein the activation of S. typhi Vi capsular polysaccharide is carried out in the presence of 2-(N-morpholino)ethanesulfonic acid (MES) in the presence of I-ethyl-3-(3-dimethylaminopropyl) carbodiimide (EDAC).

7. The process as claimed in claim 3, wherein I-ethyl-3-(3-dimethylaminopropyl) carbodiimide is in the range of 1:2 to 1: 5 w/w in Vi solution.

8. The process as claimed in claim 3, wherein the conjugation reaction is carried out in pH range of 5.4 to 5.8.

9. The process as claimed in claim 3, wherein the conjugation of flagellin to Vi polysaccharide occurs at carboxylic group through cross-linking agent.

10. The process as claimed in claim 3, wherein the cross-linking agent is selected from the group including adipic acid dihydrazine, carbodiimides such as I-cyclohexyl-3-(2-morpholinyl-(4-ethyl)carbodiimide (CMC), I-ethyl-3-(3-dimethylaminopropyl)carbodiimide (EDC), and I-ethyl-3-(4-azonia-44-dimethylpentyl)carbodiimide; cyanogen bromide, glutaraldehyde and succinic anhydride.

11. The process as claimed in claim 3, wherein the homobifunctional agent is selected from the group including bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfbydryl-reactive cross-linkers 1,4-di-[3-(2-pyridyldithio)propionamido]butane, bismaleimidohexane, and bis-N-maleimido-1,8-octane; the bifunctional aryl halides I,5-difluoro-2,4-dinitrobenzene and 4,4-difluoro-3,3-dinitrophenylsulfone; bifunctional photoreactive agents such as bis-[b-(4-azidosalicylamide)ethyl]disulfide; the bifunctional aldehydes formaldehyde, malondialdehyde, succinaldehyde, glutaraldehyde, and adiphaldehyde; a bifunctional epoxied such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides NIN-ethylene-bis(iodoacetamide), NIN-hexamethylene-bis(iodoacetamide), NIN-undecamethylene-bis(iodoacetamide), as well as benzylhalides and halomustards, such as ala-diiodo-p-xylene sulfonic acid and tri(2-chloroethyl)amine, respectively, preferably the reagent is adipic acid dihydrazide (ADH) and EDAC in MES (morpholino ethane sulphonic acid) buffer.

12. A composition comprising the conjugate as claimed in claim 1.

13. A composition comprising the conjugate as claimed in claim 12, wherein the composition is entrapped in polymer nanoparticles selected from the group including polylactide, polyglycolide, polylactide-co-glycolide, polycaprolactone, polyhydroxyacid, preferably polylactic acid nanoparticles.

14. A process for preparation of nanoparticle composition as claimed in claim 12 comprises the steps of:
(i) dissolving the conjugate in a solution;
(ii) adding an emulsifier such as polylactic acid (PVA);
(iii) adding an organic phase comprising solvents such as dichloromethane and sonification to form a primary (W/0) emulsion; and
(iv) Further emulsifying the primary emulsion from step (iii) to obtain a secondary (W/0/W)emulsion.

15. A process for preparation of nanoparticle composition as claimed in claim 14, wherein the process comprising entrapping conjugate in polylactic acid nanoparticles is done by a double emulsion solvent evaporation method.

16. The process as claimed in claim 14, including dissolving the components of the composition in aqueous phases containing 0.5 to 1% (w/v) polyvinylalchol as emulsifier.

17. The process as claimed in claim 14, including adding the aqueous phase in dichloromethane (DCM) organic phase containing polylactic acid polymer with sonication and forming a primary emulsion.

18. The process as claimed in claim 14, wherein the primary emulsion is added to external aqueous phase containing polyvinylalchol (PVA) as surfactants with sonication to form water in oil in water secondary emulsion.

19. Use of conjugate as claimed in claim 1 for immunization against typhoid and as single dose administration for immunization againsttyphoid.
